(19) United States
(12) Patent Application Publication
(10) Pub. No.: US 2011/0015525 A1
(43) Pub. Date: Jan. 20, 2011

(54) ULTRASONOGRAPH

(75) Inventors: Hiroshi Fukukita, Tokyo (JP); Yoshihiko Itoh, Kanagawa (JP)

Correspondence Address: HAMRE, SCHUMANN, MUELLER & LARSON P.C., P.O. BOX 2902, MINNEAPOLIS, MN, 55402-0902, US

(73) Assignee: PANASONIC CORPORATION, Kadoma-shi, Osaka (JP)

(21) Appl. No.: 12/921,304

(22) PCT Filed: Mar. 3, 2009
(86) PCT No.: PCT/JP2009/000948

§ 371 Date: Sep. 7, 2010

(30) Foreign Application Priority Data
Mar. 7, 2008 (JP) 2008-057460

(51) Int. Cl. A61B 8/14

(52) U.S. Cl. 600/447

(57) ABSTRACT

Provided is an ultrasonograph that scans a subject in a region of two or more dimensions. The ultrasonograph includes an array transducer (1) in which a plurality of transducers (2) are arrayed, wherein the array transducer (1) is divided into a plurality of sub-arrays (3, 4) composed of a plurality of adjacent ones of the transducers (2). Outputs of the transducers (2) constituting the sub-arrays (3, 4) are input to switch arrays (5, 6) corresponding to the sub-arrays (3, 4), respectively, and outputs of the switch arrays (5, 6) are input via tap input sample hold amplifiers of delay addition lines (7, 8) to sample-hold stages of the delay addition lines (7, 8). The delay addition lines (7, 8) have a configuration in which the plurality of sample-hold stages are connected in series. Outputs of the delay addition lines (7, 8) are the sum of outputs from the transducers (2) constituting the same sub-arrays (3, 4).

### TECHNICAL FIELD

The present invention relates to an ultrasonograph that has an array probe in which a plurality of transducers are arrayed and scans a subject in a region of two or more dimensions.

### BACKGROUND ART

As conventional ultrasonographs, an ultrasonograph has been known in which, in order to delay and add reception signals in reception sub-arrays constituting an array transducer, in-group reception processors are connected to the reception sub-arrays. This in-group reception processor includes a sample-hold circuit and some adding elements that are arranged so as to form a delay addition line. Further, as such in-group reception processors, there is one in which a cross point switch is arrayed so as to connect a signal between a transducer element and a tap selected with a delay addition line (see Patent Document 1, for example).

[Patent Document 1] JP 2000-33087 A (paragraphs [0122], [0125] and [0126])

### DISCLOSURE OF INVENTION

### Problem to be Solved by the Invention

However, in the conventional ultrasonograph, since a reception signal is added to a sampled-held signal, an input signal has varied due to an influence of the reception signal when the sample-hold circuit in the next stage samples the input signal. This has caused a problem in that it is difficult for the sample-hold circuit in the next stage to perform a sample-hold operation accurately.

The present invention has been made to solve the foregoing problem of the conventional ultrasonograph, and it is an object of the present invention to provide an ultrasonograph capable of operating sample-hold circuits constituting delay addition lines accurately.

### Means for Solving Problem

An ultrasonograph of the present invention includes an array transducer in which a plurality of transducers are arrayed, wherein the array transducer is divided into a plurality of sub-arrays composed of a plurality of adjacent ones of the transducers; an output of each of the transducers constituting the sub-array is input to a switch array corresponding to the sub-array, and an output of the switch array is input via a tap input sample-hold amplifier of a delay addition line to a sample-hold stage of the delay addition line; the delay addition line has a configuration in which a plurality of the sample-hold stages are connected in series; and an output of the delay addition line is the sum of outputs from the transducers constituting the same sub-array.

With this configuration, since a signal to be input to the sample-hold stage of the delay addition line is held by the tap input sample-hold amplifier, the value of the signal to be input to the sample-hold stage of the delay addition line can be stabilized. Thereby it is possible to operate the sample-hold stage of the delay addition line accurately.

Further, as to the ultrasonograph of the present invention, it is preferable that the sample-hold stage has a configuration in which a sample-hold pre-amplifier and a sample-hold post-amplifier are connected in series. With this configuration, the sample-hold stage can transmit a reception signal to the sample-hold stage in the next stage accurately.

Further, as to the ultrasonograph of the present invention, it is preferable that the sample-hold pre-amplifier is a current input and a voltage output, and the sample-hold post-amplifier is a voltage input and a current output. With this configuration, the sample-hold stage can be a current input and a current output, and hence, the input and output thereof with respect to other circuits are performed in a current mode. Consequently, it is possible to obtain a high-speed sample-hold stage having high exogenous noise resistance.

Further, it is preferable that the tap input sample-hold amplifier is a current output. With this configuration, it becomes easier to inject signals to the sample-hold stage operating in the current mode.

Furthermore, it is preferable that the tap input sample-hold amplifier and the sample-hold post-amplifier operate in synchronization with each other. With this configuration, a reception signal can be input to the sample-hold stage with accurate timing.

### EFFECT OF THE INVENTION

An ultrasonograph of the present invention includes an array transducer in which a plurality of transducers are arrayed, wherein the array transducer is divided into a plurality of sub-arrays composed of a plurality of adjacent ones of the transducers, an output of each of the transducers constituting the sub-array is input to a switch array corresponding to the sub-array, and an output of the switch array is input via a tap input sample-hold amplifier of a delay addition line to a sample-hold stage of the delay addition line. The delay addition line has a configuration in which a plurality of the sample-hold stages are connected in series, and an output of the delay addition line is the sum of outputs from the transducers constituting the same sub-array. With this configuration, since a signal to be input to the sample-hold stage of the delay addition line is held by the tap input sample-hold amplifier, the value of the signal to be input to the sample-hold stage of the delay addition line can be stabilized. Thus, the present invention can provide an ultrasonograph having an effect of accurately operating the sample-hold stages of the delay addition line.

### BRIEF DESCRIPTION OF DRAWINGS

FIG. 1 is a block diagram showing an overall configuration of an ultrasonograph in an embodiment of the present invention.

FIG. 2 is a block diagram showing a detailed configuration of a switch array of the ultrasonograph in the embodiment of the present invention.

FIG. 3 is a block diagram showing a configuration of a delay addition line of the ultrasonograph in the embodiment of the present invention.

FIG. 4 is a block diagram showing a detailed configuration of the delay addition line of the ultrasonograph in the embodiment of the present invention.

FIG. 5 is a time chart showing clock waveforms of the delay addition line of the ultrasonograph in the embodiment of the present invention.

FIG. 6 is a block diagram showing a detailed configuration of a sample-hold pre-amplifier of the ultrasonograph in the embodiment of the present invention.

FIG. 7 is a block diagram showing a detailed configuration of a sample-hold post-amplifier of the ultrasonograph in the embodiment of the present invention.

### DESCRIPTION OF REFERENCE NUMERALS

1 array transducer
2 transducer
3, 4 sub-array
5, 6 switch array
7, 8 delay addition line
71, 72, 73 sample-hold stage
74, 75, 76, 77 tap input sample-hold amplifier
81, 83, 85 sample-hold pre-amplifier
82, 84, 86 sample-hold post-amplifier

### BEST MODE OF CARRYING OUT THE INVENTION

Hereinafter, an ultrasonograph according to an embodiment of the present invention will be described using the drawings.

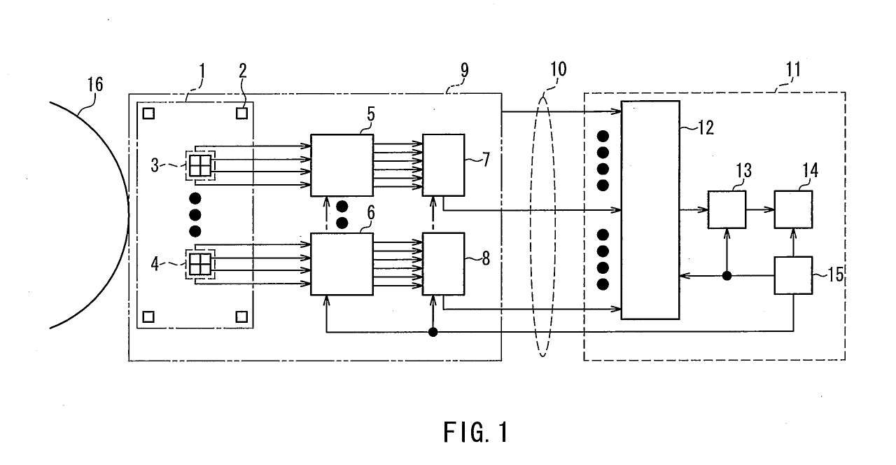

FIG. 1 is a block diagram showing an overall configuration of the ultrasonograph according to the embodiment of the present invention.

As shown in FIG. 1, an array transducer 1 is composed of a plurality of transducers 2 that are arrayed in two-dimensional directions (row and column directions), each direction including a plural number of transducers 2. The transducers 2 constituting the array transducer 1 form a plurality of sub-arrays (3, 4) made of two or more adjacent transducers 2. Thus, the array transducer 1 is divided into the plurality of sub-arrays (3, 4). For the sake of simplicity of the drawing, only a first sub-array 3 and a second sub-array 4 are illustrated in FIG. 1.

Note here that FIG. 1 illustrates, as the array transducer 1, a matrix array in which the plurality of transducers 2 are arrayed in the row and column directions, but the array transducer 1 of the ultrasonograph of the present invention is not limited by the arrangement mode of the transducers 2. Therefore, as the array transducer 1 of the ultrasonograph of the present invention, a linear array in which transducers are arrayed in one-dimensional direction, a convex array and the like are applicable, other than the matrix array shown in FIG. 1.

Outputs of the first sub-array 3 are input to a first switch array 5, and outputs of the second sub-array 4 are input to a second switch array 6. Outputs of the first switch array 5 are input to a first delay addition line 7, and outputs of the second switch array 6 are input to a second delay addition line 8. Note here that, since FIG. 1 only illustrates the first sub-array 3 and the second sub-array 4 as sub-arrays, only two each (first and second) of the switch arrays and the delay addition lines are illustrated. However, naturally, the ultrasonograph of the present embodiment includes the numbers of the switch arrays and the delay addition lines that correspond to the number of the sub-arrays.

The aforementioned array transducer 1, the plurality of switch arrays (5, 6), and the plurality of delay addition lines (7, 8) are housed in a probe handle 9.

Outputs of the delay addition lines (7, 8) are input via cables 10 to a main beam former 12 of a main body 11 of the ultrasonograph. The main beam former 12 can be oriented simultaneously to multiple directions so as to obtain a plurality of reception signals. A plurality of outputs from the main beam former 12 are signal-processed at a signal processing part 13 to be displayed on a display part 14.

A control part 15 controls the switch arrays (5, 6), the delay addition lines (7, 8), the main beam former 12, the signal processing part 13, the display part 14, etc.

The array transducer 1 is brought into contact with a subject 16.

Next, a flow of the signal processing of reception signals in the aforementioned ultrasonograph of the present embodiment will be described in detail.

In FIG. 1, an ultrasonic pulse is sent out from the array transducer 1, and the ultrasonic pulse reflected at the subject 16 is received by the sub-arrays (3, 4) of the array transducer 1. Reception signals received by the respective transducers 2 of the first sub-array 3 are input via the first switch array 5 to any one of inputs of the first delay addition line 7. Similarly, reception signals received by the respective transducers 2 of the second sub-array 4 are input via the second switch array 6 to any one of inputs of the second delay addition line 8.

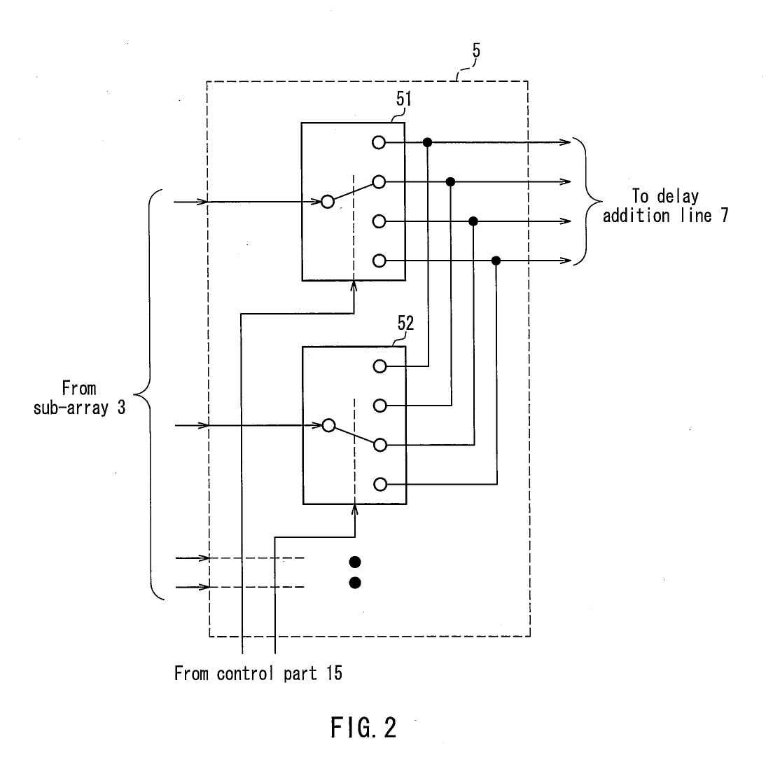

FIG. 2 shows a more detailed configuration of the switch array of the ultrasonograph according to the present embodiment, and is a block diagram showing a configuration of the first switch array 5.

As shown in FIG. 2, the first switch array 5 has a plurality of demultiplexers (51, 52). For example, in the demultiplexer 51, the reception signal from the first sub-array 3 passes through an output terminal selected by a control signal from the control part 15 and is input to the first delay addition line 7.

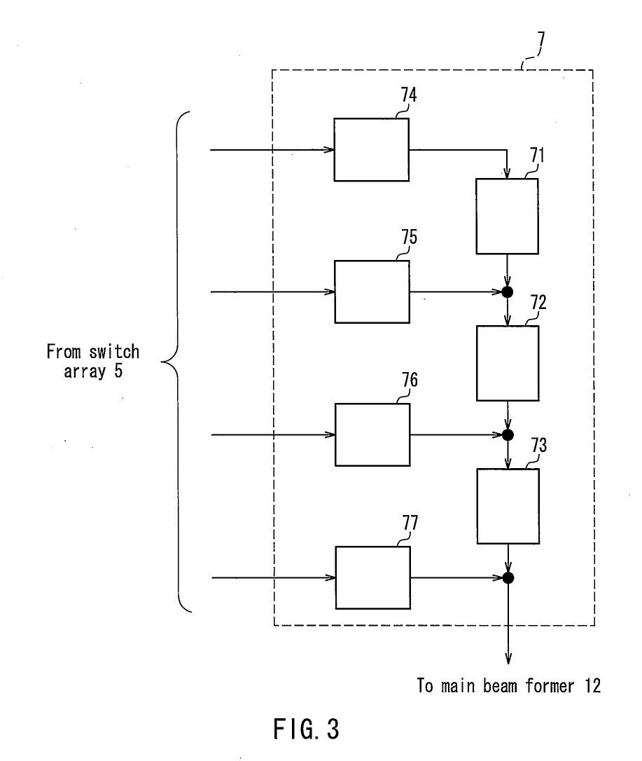

FIG. 3 shows a more detailed configuration of the delay addition line of the ultrasonograph according to the present embodiment, and is a block diagram showing a configuration of the first delay addition line 7.

As shown in FIG. 3, the first delay addition line 7 includes sample-hold stages (71, 72, 73) and tap input sample-hold amplifiers (74, 75, 76, 77). The reception signals from the respective transducers 2 of the first sub-array 3 having passed through the first switch array 5 are input to any one of the tap input sample-hold amplifiers (74, 75, 76, 77) and added at the sample-hold stages (71, 72, 73) so as to be an output of the first delay addition line 7. The output of the first delay addition line 7 is input to the main beam former 12.

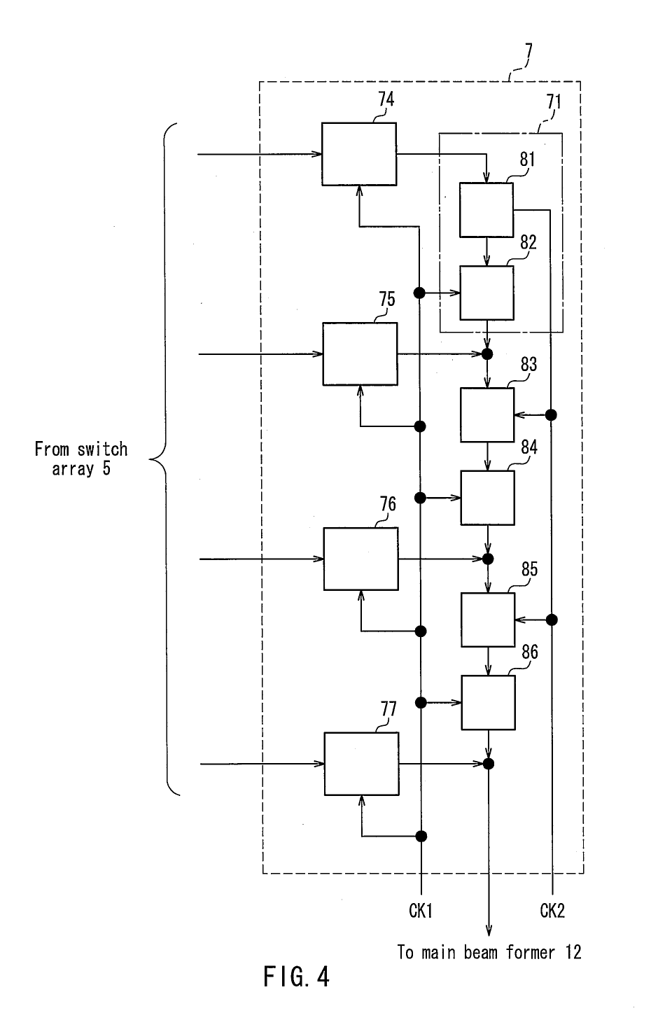

FIG. 4 shows a further detailed configuration of the delay addition line of the ultrasonograph according to the present embodiment, and is a block diagram showing a more detailed configuration of the first delay addition line 7.

As shown in FIG. 4, the sample-hold stage 71 of the first delay addition line 7 includes a sample-hold pre-amplifier 81 and a sample-hold post-amplifier 82. An output of the tap input sample-hold amplifier 74 is input to the sample-hold pre-amplifier 81, and an output of the sample-hold pre-amplifier 81 is input to the sample-hold post-amplifier 82.

A first clock signal CK1 controls the tap input sample-hold amplifiers (74, 75, 76, 77) and the sample-hold post-amplifiers (82, 84, 86). A second clock signal CK2 controls the sample-hold pre-amplifiers (81, 83, 85). Therefore, the tap input sample-hold amplifiers (74, 75, 76, 77) and the sample-hold post-amplifiers (82, 84, 86) operate in synchronization with each other.

With this configuration, in the delay addition lines (7, 8) in the ultrasonograph of the present embodiment, reception signals can be input to the sample-hold stages (71, 72, 73) with accurate timing.

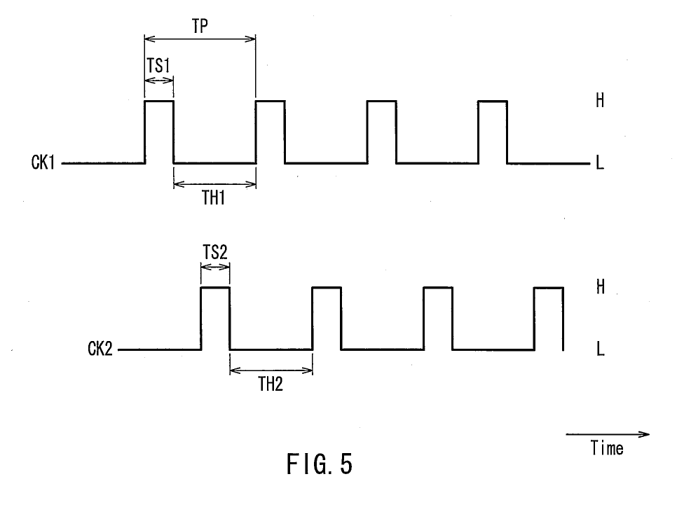

FIG. 5 is a time chart showing a timing relationship between the first clock signal CK1 and the second clock signal CK2 of the ultrasonograph in the present embodiment.

As shown in FIG. 5, while a theoretical value of the first clock signal CK1 is in an H period TS1, each of the sample-hold amplifiers controlled by the first clock signal CK1 follows the input. While the theoretical value of the first clock signal CK1 is in an L period TH1, they hold the input value.

Meanwhile, a theoretical value of the second clock signal CK2 is in an H period TS2 when the theoretical value of the first clock signal CK1 is in the L period. While this theoretical value is in the H period TS2, the sample-hold pre-amplifiers follow the value held by the sample-hold amplifiers in the previous stage.

Therefore, the reception signal from the switch array is held by the tap input sample-hold amplifier 75 and then is added to an output held by the sample-hold post-amplifier 82. Because of this, in the period TS2 in which the sample-hold pre-amplifier 83 in the next stage samples an input signal, an input signal is held, which allows the sample-hold pre-amplifier 83 to sample the input value accurately.

The sum of the periods TS1 and TH1 is a sampling interval TP. When a period of one cycle of the input signal is assumed to be TC, TP is equal to TC/N (N=about 8 to 16, for example). Further, a delay time of the first delay addition line 7 may be set at about 2TC.

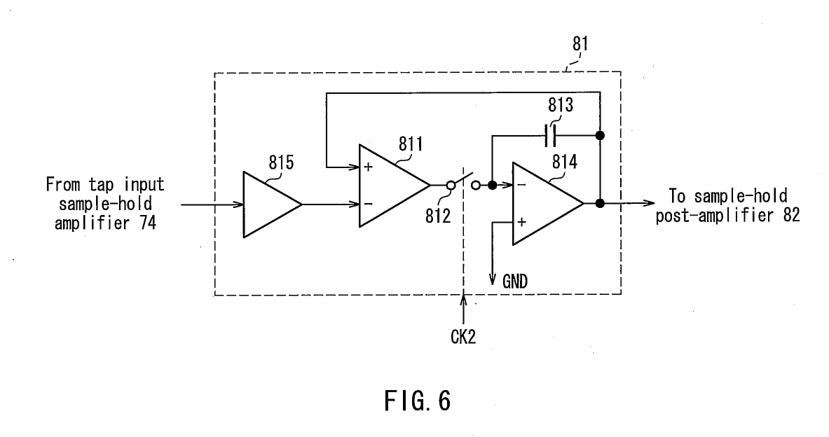

FIG. 6 is a block diagram showing a detailed configuration of the sample-hold pre-amplifier 81 of the ultrasonograph in the present embodiment.

As shown in FIG. 6, a current output of the tap input sample-hold amplifier 74 is input to a current input/voltage output amplifier 815, and an output of the amplifier 815 is input to an inverting input of an amplifier 811, and an output of the amplifier 811 is input via a switch 812 to an inverting input of an amplifier 814. Note here that a non-inverting input of the amplifier 814 is connected to GND. Further, a capacitor 813 is disposed between the inverting input and an output of the amplifier 814.

Further, the output of the amplifier 814 is connected with a non-inverting input of the amplifier 811, which allows the sample-hold pre-amplifier 81 to be a voltage output.

When the theoretical value of the second clock signal CK2 is in the H period, the switch 812 is turned on, whereby the sample-hold pre-amplifier 81 follows the input signal. Meanwhile, when the theoretical value of the second clock signal CK2 is in the L period, the switch 812 is turned off, whereby the input signal is held.

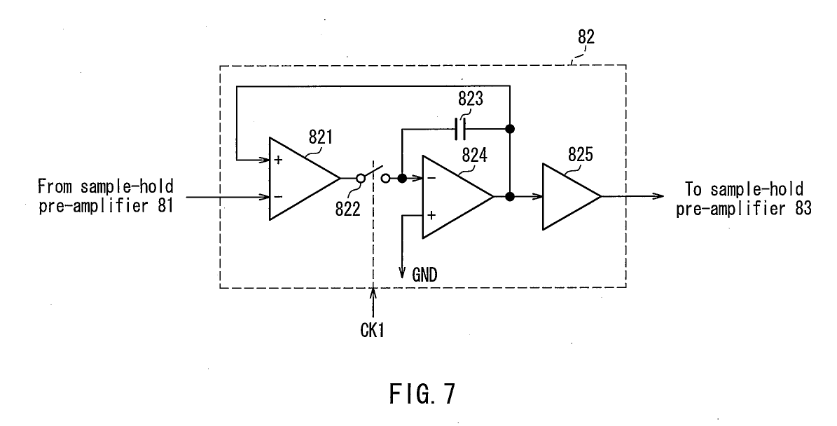

FIG. 7 is a block diagram showing a detailed configuration of the sample-hold post-amplifier 82 of the ultrasonograph in the present embodiment. Note here that the tap input sample-hold amplifier 74 has a configuration similar to the configuration of the sample-hold post-amplifier 82 shown in FIG. 7.

As shown in FIG. 7, the voltage output of the sample-hold pre-amplifier 81 is input to an inverting input of an amplifier 821, and an output of the amplifier 821 is input via a switch 822 to an inverting input of an amplifier 824. A capacitor 823 is disposed between the inverting input and an output of the amplifier 824. Further, the output of the amplifier 824 is connected with a non-inverting input of the amplifier 821.

When the theoretical value of the first clock signal CK1 is in the H period, the switch 822 is turned on, whereby the sample-hold post-amplifier 82 follows the input signal. Meanwhile, when the theoretical value of the first clock signal CK1 is in the L period, the switch 822 is turned off, whereby the input signal is held.

The output of the amplifier 824 is input to a voltage input/current output amplifier 825, which allows an output of the sample-hold post-amplifier 82 to be a current output.

Further, since the tap input sample-hold amplifier 75 having a configuration shown in FIG. 7 is a current output likewise the sample-hold post-amplifier 82, the current output of the tap input sample-hold amplifier 75 is added to the current output of the sample-hold post-amplifier 82 and then is input to the sample-hold pre-amplifier 83 with a current input.

Thus, by setting the outputs of the tap input sample-hold amplifiers (74, 75, 76, 77) to be a current-mode output, signals can be added speedily with high precision. Note here that a current gain of the sample-hold stages in this case is one time.

The delayed-added outputs of the sub-arrays (3, 4) thus obtained are input via the cables 10 to the main beam former 12, and the received delayed-added outputs of the main beam former 12 which are parallel-processed and have a plurality of directivities then are signal-processed at the signal processing part 13 to be displayed on the display part 14.

The above-described ultrasonograph according to the embodiment of the present invention includes an array transducer in which a plurality of transducers are arrayed, wherein the array transducer is divided into a plurality of sub-arrays composed of a plurality of adjacent ones of the transducers, an output of each of the transducers constituting the sub-array is input to a switch array corresponding to the sub-array, and an output of the switch array is input via a tap input sample-hold amplifier of a delay addition line to a sample-hold stage of the delay addition line. The delay addition line has a configuration in which a plurality of the sample-hold stages are connected in series, and an output of the delay addition line is the sum of outputs from the transducers constituting the same sub-array. With this configuration, since a signal to be input to the sample-hold stage of the delay addition line is held by the tap input sample-hold amplifier, the value of the signal to be input to the sample-hold stage of the delay addition line can be stabilized. Thus, the present invention can provide an ultrasonograph having an effect of accurately operating the sample-hold stages of the delay addition line.

### INDUSTRIAL APPLICABILITY

As described above, the ultrasonograph according to the present invention has an effect of accurately performing a delay addition operation with respect to reception signals of each sub-array of the array transducer, and has an array probe in which a plurality of transducers are arrayed, and therefore, is industrially useful as an ultrasonograph or the like that scans a subject in a region of two or more dimensions.

1. An ultrasonograph comprising an array transducer in which a plurality of transducers are arrayed,
wherein the array transducer is divided into a plurality of sub-arrays composed of a plurality of adjacent ones of the transducers,
an output of each of the transducers constituting the sub-array is input to a switch array corresponding to the sub-array, and an output of the switch array is input via a tap input sample-hold amplifier of a delay addition line to a sample-hold stage of the delay addition line,
the delay addition line has a configuration in which a plurality of the sample-hold stages are connected in series, and
an output of the delay addition line is the sum of outputs from the transducers constituting the same sub-array.

2. The ultrasonograph according to claim 1, wherein the sample-hold stage has a configuration in which a sample-hold pre-amplifier and a sample-hold post-amplifier are connected in series.

3. The ultrasonograph according to claim 2, wherein the sample-hold pre-amplifier is a current input and a voltage output, and the sample-hold post-amplifier is a voltage input and a current output.

4. The ultrasonograph according to claim 3, wherein the tap input sample-hold amplifier is a current output.

5. The ultrasonograph according to claim 4, wherein the tap input sample-hold amplifier and the sample-hold post-amplifier operate in synchronization with each other.
